(19) United States
(12) Patent Application Publication
(10) Pub. No.: US 2005/0170503 A1
(43) Pub. Date: Aug. 4, 2005

(54) In vitro induction of antigen-specific T-cells using dendritic cell-tumor cell or dendritic cell-viral cell derived immunogens

(75) Inventors: Louis D. Falo JR., Wexford, PA (US); Walter Storkus, Glenshaw, PA (US)

Correspondence Address: FOLEY AND LARDNER;SUITE 500, 3000 K STREET NW, WASHINGTON, DC, 20007, US

(73) Assignee: University of Pittsburgh

(21) Appl. No.: 11/090,122

(22) Filed: Mar. 28, 2005

Related U.S. Application Data
(63) Continuation of application No. 09/282,679, filed on Mar. 31, 1999, now abandoned.
Application No. 09/282,679 is a continuation-in-part of application No. 09/030,985, filed on Feb. 26, 1998, now abandoned.
(60) Provisional application No. 60/039,472, filed on Feb. 27, 1997.

(51) Int. Cl. C12N 5/08; C12N 15/07; C12N 15/867

(52) U.S. Cl. 435/372; 435/456; 435/451

(57) ABSTRACT

Antigen-specific T-cells prepared by culturing T-cells in formulations comprising combinations of DCs and either tumor cells or virally infected cells are disclosed. These formulations generally comprise hybridoma of at least one dendritic cell fused to either at least one tumor cell or at least one virally infected cell, or co-cultures of dendritic cells and either tumor cells or virally infected cells. The resulting T-cells can then be used in immunotherapy methods through adoptive transfer of autologous antigen-specific T-cells into patients using well-established techniques, as agents to identify tumor antigens, and to establish animal models.

### CROSS REFERENCE TO RELATED APPLICATIONS

This application is a continuation-in-part of U.S. Ser. No. 09/030,985 filed Feb. 26, 1998, which claims priority to U.S. Ser. No. 60/039,472, filed Feb. 27, 1997.

### FIELD OF THE INVENTION

The present invention generally relates to antigen-specific T-cells and methods for making and using the same. More specifically, the present invention relates to antigen-specific T-cells that have been generated by co-culture with immunogens derived from a formulation comprising either hybridomas of dendritic cells and tumor cells or co-culture products of dendritic cells and tumor cells. Alternatively, virally infected cells can be used instead of tumor cells in these hybridomas and co-cultures. The use of these T-cells as prophylactic and therapeutic agents against tumors and viral infection is also the subject of the present invention.

### BACKGROUND OF THE INVENTION

T-cells, including cytotoxic T-lymphocytes (CTLs), are a critical component of effective human immune responses to tumors and viral infections. T-cell responses are sufficient to protect against tumors and viruses and can eliminate even established cancers in murine tumor models and in humans. T-cells destroy neoplastic cells or virally infected cells through recognition of antigenic peptides presented by MHC Class I molecules on the surface of the effected target cells. These antigenic peptides are degradation products of foreign proteins present in the cytosol of the effected cell, which are processed and presented to T-cells through the endogenous MHC Class I processing pathway. CTLs target tumors through recognition of a ligand consisting of a self MHC Class I molecule and a peptide antigen. The development of CTL-dependent anti-tumor immunization strategies, therefore, typically depends on both the identification of tumor antigens recognized by CTLs and the development of methods for effective antigen delivery.

Although the recognition of a foreign protein in the context of the MHC Class I molecule may be sufficient for the recognition and destruction of effected target cells by CTLs, the induction of antigen-specific CTLs from T-lymphocyte precursors requires additional signals. Specialized antigen-presenting cells (APCs) can provide both the antigen MHC Class I ligand and the accessory signals required in the induction phase of CTL mediated immunity. General properties of APCs include MHC Class I and Class II expression, expression of various adhesion molecules important for APC-lymphocyte interaction, and expression of co-stimulatory molecules such as CD80 and CD86. APCs include, for example, macrophages, B-cells, and dendritic cells, including cutaneous epidermal Langerhans cells, dermal dendritic cells, and dendritic cells resident in lymph nodes and spleen. Dendritic cells (DCs) are believed to be the most potent APCs, and can induce effective CTL-dependent anti-tumor immunity. Procedures are available to obtain significant quantities of dendritic cells from bone marrow or peripheral blood derived precursors.

It is likely that a tumor cell expresses a set of tumor-specific peptide MHC complexes which can be recognized by CTLs. Progressive tumors, however, are generally non-immunogenic, at least in part, because they are incapable of providing co-stimulation.

Guo et al., Science 263: 518-520 (1994), disclose tumor vaccines generated by fusion of hepatoma cells with activated B-cells. The fusion of the activated B-cells and the tumor cells produce an immunogen capable of inducing tumor-specific protective tumor immunity.

Mayordomo et al., Nature Med. 1 (12): 1297-1302 (1995), disclose in vitro culture of peptide-pulsed dendritic cells that show protection against the associated tumor challenge. The dendritic cells cultured in the presence of GM-CSF+ILA and transfected with chicken ovalbumin (OVA) were capable of preventing establishment of an OVA+tumor, but not the untransfected parental melanoma.

Flamand et al., Eur. J. Immunol. 24: 605-610 (1994), disclosed in vitro culture of dendritic cells pulsed with a peptide antigen BCL1, and subsequent induction of a T-cell dependent humoral response to the B-cell tumor BCL1. A similar methodology is reported by Celluzzi et al., J. Exp. Med., 183: 203-287 (1996). There, MHC I-peptide antigens were pulsed onto dendritic cells; immunized hosts showed protective immunity to a lethal challenge by a tumor transfected with the antigen gene.

Hsu et al., Nature Medicine 2: 52-58 (1996) investigated the use of dendritic cells pulsed with tumor-specific idiotype proteins as vaccines.

Celluzzi and Falo, J. Immunol. p. 3081-3085 (1998), disclose formulations comprising dendritic cells fused to or co-cultured with tumor cells.

Gong et al., Nature Med. 3:558 (1997), disclose fusion of dendritic cells with MC38 carcinoma cells.

U.S. Pat. No. 5,788,963 discloses preparation of dendritic cells for prostate cancer immunotherapy; stimulation of antigen specific T-cells by the dendritic cells is reported. The patent appears to be limited to exposure of dendritic cells to specific, defined prostate cancer antigens, or to lysates or fractionated lysates as potential sources for these antigens. The patent methodologies do not appear to include fusion or co-culture of dendritic cells with whole tumor cells or with virally infected cells, or the use of undefined antigens.

U.S. Pat. No. 5,846,827 reports methods for using peptide-loaded antigen presenting cells for the activation of CTL. The methods appear to rely on the modification of antigen presenting cells in vivo by exposure to specific identified and isolated epitopes. There does not appear to be any teaching of the antigen specific T-cells or methods of producing these T-cells as taught below.

None of the above articles or patents appear to teach or suggest the generation of a unique, antigen-specific T-cell such as those disclosed herein. In addition, none of these offer a significant advantage of the present invention, namely the elimination of the need to isolate and/or identify specific antigens.

There is a need for cancer immunotherapy that provides protective and therapeutic immunity to a wide variety of tumor types. A similar need exists for viral immunotherapy directed towards a wide variety of viral infections.

### SUMMARY OF THE INVENTION

The present invention has met the above described needs by providing antigen-specific T-cells and methods of making and using the same. Generally, the Teells are prepared by co-culture of T-ells with formulations comprising dendritic cells and either tumor cells or virally-infected cells. One embodiment of this invention uses a formulation comprising one or more hybridomas; each hybridoma is further comprised of at least one dendritic cell fused to at least one of either a tumor cell or a virally-infected cell. Another embodiment of this invention uses a formulation comprising the products of co-culture of dendritic cells and either tumor cells or virally-infected cells. Both of these formulations produce immunogens specific for the type of tumor or virus used in the formulation. Co-culture of either of these formulations with T-cells results in T-cells that are antigen-specific; the generated T-cells will recognize, and attack, cells expressing the particular antigens with which the T-cells have been co-cultured. In the case of tumor immunotherapy, the generated T-cells provide both protection against tumor challenge and regression of tumor growth. Thus, the T-cells of the present invention provide prophylactic resistance to tumors of the type represented by the tumor cell used in the formulation, and also provide a therapeutic treatment for patients suffering from such tumors. Similarly, in the case of viral immunotherapy, the generated T-cells protect against the viral infection caused by the virally infected cells used in the formulation, and/or provide therapeutic relief for patients having such viral infections.

Tumor cells and virally infected cells express antigens which can be targeted by T-cells, but the tumor cells and virally infected cells themselves do not stimulate T-cell immunity. This is presumably because the tumor cells and viral cells are incapable of providing the antigen or antigens in the appropriate context of co-stimulation. Antigen presenting cells (APC), of which dendritic cells (DC) are thought to be the most potent, express a variety of co-stimulatory molecules and cytokines. The present invention uses formulations in which DCs are fused to or are in a co-culture with either tumor cells or virally infected cells. Fusion or co-culture of the DCs with the tumor cells or virally infected cells causes the antigens to become more immunogenic by association with the DCs, which are “professional” antigen presenting cells. The fusion products and the co-culture products express properties of both the DC and either the tumor or virus. The fused cells and/or co-cultured cells are then further co-cultured with unstimulated T-cells. During this co-culture, the T-cell is exposed to a complete array of antigens from either the tumor cells or virally-infected cells. The generated T-cells which result are then antigen-specific, and will destroy tumor cells that express the same or similar tumor antigens or virally-infected cells that express the same or similar viral antigens.

As will be appreciated by one skilled in the art, therefore, the present invention obviates the need to identify specific antigens that elicit a T-cell response by providing T-cells that are generated or engineered to recognize the antigens with which they are co-cultured. By delivering and co-culturing the entire array of antigens produced by a tumor cell or a virally infected cell with the T-ells, a mechanism is provided for broad, polyvalent immunotherapy.

It is therefore an object of the present invention to provide antigen-specific T-cells.

It is a further object of the present invention to provide a pharmaceutical composition comprised of antigen-specific T-cells.

It is a further object of the present invention to provide methods for treatment of a patient with antigen-specific T-cells.

Yet another object of the invention is to provide methods for the prophylactic and/or therapeutic treatment of cancer and viral infections.

Another object of the present invention is to provide antigen-specific T-cells which can be used to identify tumor or viral antigens.

These and other objects of the invention will be more fully understood from the following description of the invention.

### BRIEF DESCRIPTION OF THE DRAWINGS

FIG. 1 illustrates flow labelling patterns showing the efficiency of association between tumor cell components and dendritic cells, as described in Example 3.

FIG. 2 shows the % specific lysis of peptide loaded cells, as described in Example 4.

FIG. 3 shows the % lysis of tumor cells, as described in Example 4.

### DETAILED DESCRIPTION OF THE INVENTION

The present invention is directed to methods for generating tumor antigen-specific T-cells by combining dendritic cells from a patient with tumor cells, preferably from the same patient, in vitro; adding T-cells from the patient to the combination of DCs and tumor cells; culturing the T-cell/DC/tumor cell mixture; and harvesting the T-cells from the co-culture. The present invention is further directed to similar methods using virally-infected cells rather than tumor cells, which would result in viral antigen-specific T-cells. T-cells prepared according to either of these methods are also within the scope of the present invention. The T-cells prepared according to the present invention have demonstrated efficacy in providing both prophylactic and therapeutic relief to patients at risk for, or suffering from, tumors or viral infections.

The first step of the present methods involves combining dendritic cells from a patient with either tumor cells or virally-infected cells. Preferably, the tumor cells or virally infected cells are from the same patient. The cells can be combined in any manner known in the art. Preferred for use in the present methods are fusions of the DC with the afflicted cell and co-culture of the DC with the afflicted cell. As used herein, the terms “afflicted cell” or “afflicted cells” refer generally to either tumor cells or virally infected cells; the afflicted cells will vary from application to application depending on the particular type of T-cell that is to be generated and the type of immunotherapy desired.

In one embodiment, therefore, the DC and afflicted cell are fused to form a hybridoma. As will be appreciated by those skilled in the art, a hybridoma is a physical combination of at least two different kinds of cells. At least two different hybridomas fall within the scope of the present invention—namely a hybridoma between at least one DC and one tumor cell, and a hybridoma between at least one DC and at least one virally infected cell.

According to one embodiment of the present invention, one or more dendritic cells are fused to one or more tumor cells to form hybridomas or fusion products in the first step of the present methods. Any starting ratio of dendritic cells to tumor cells can be used. For example, the starting ratio can be anywhere from 1:10 to 10:1, 1:100 to 100:1, or even higher. In a preferred embodiment, this fusion product is made with a starting ratio of dendritic cells to tumor cells of about 6:1; this starting ratio was found to yield a sufficient number of DC/tumor cell hybridomas. Preferably, the DC:tumor cell ratio includes a higher number of DCs, as a higher number of DCs increases the probability that the tumor cells will become fused to at least one DC. As will be appreciated by one skilled in the art, one or more DCs can become fused to one or more tumor cells. Thus, the hybridomas prepared in the first step of the methods can have a range of DC:tumor cell ratios. For example, when starting with an DC:tumor cell ratio of 6:1, the resulting hybridomas could have an DC:tumor cell ratio of anywhere from about 1:1 to 10:1 or more.

Any type of DCs can be used. DCs, it will be appreciated, are one type of antigen presenting cell (“APC”), which is a cell capable of presenting antigens. DCs are found throughout the body, and include cutaneous epidermal Langerhans cells, dermal dendritic cells, dendritic cells located in the lymph nodes and spleen, and dendritic cells derived through in vitro culture of precursors. Dendritic cells can be obtained from a host by any means known in the art. For example, dendritic cells can be obtained from bone marrow according to the methods of Celluzzi, et al., J. Exp. Med. 183: 283-287 (1996). DCs can also be obtained from peripheral blood and skin. Blood-derived DCs are the preferred DCs for use in the present invention.

Any type of tumor cells can be used, including but not limited to, tumor cells obtained from patients including melanomas, lung cancers, prostate cancers, breast cancers, colon cancers and cervical cancers. Tumor cells for which single-cell suspensions of auto-tumor may be obtained are preferred. In addition, while autologous tumor cells are preferred, allogenic tumor cell lines can also be used as the source of tumor antigen. It is well known in the art that certain tumor antigens can be found to be present in tumor cells obtained from more than one patient. These are often referred to as “shared” tumor antigens. In the present invention, allogenic tumor cells can be used as a source of antigen as these cells can contain “shared” tumor antigens also present in the patient's tumor. For use in the present invention, the tumor cells can be treated before or after use in the present formulations by, for example, radiation or similar treatment.

According to another embodiment of the present invention, one or more DCs are fused to one or more virally infected cells. As with the DC/tumor cell fusion, any starting ratio of DCs to virally infected cells can be used. Preferably, this ratio is sufficient to allow for the fusion of all of the virally infected cells to one or more DC. Any virally infected cells can be used, including but not limited to, cells infected with influenza virus, human immunodeficiency virus (HIV), cytomegalovirus (CMV), human papilloma virus (HPV) and herpes simplex virus (HSV).

The hybridomas or fusion products of the present invention can be formed by any method known in the art. In a preferred embodiment, the DC-tumor cell or DC-virally infected cell hybridoma is formed by fusing the two types of cells together with polyethylene glycol (PEG). Generally, this method involves adding DCs and afflicted cells to the same container and centrifuging the cell suspensions to form a pellet. Approximately 1 ml of a 50% PEG solution heated to about 37° C. should be gradually added to the pellet. The pellet/PEG is gradually diluted with PBS while gentle stirring is applied. The fused cells are then washed by centrifugation and the supernatant decanted, to form the fusion product.

The first step in the methods of the present invention can also be achieved by combining DCs and afflicted cells in vitro, and co-culturing the mixture, rather than fusing the two cell types together. As used herein, “co-culture” refers to the culturing together of at least two different types of cells, here DCs and afflicted cells alone or in further combination with unstimulated T-cells. A co-culture of DCs and either tumor cells or virally infected cells can be prepared by simply culturing the DCs with either the tumor or virally infected cells; that is, the two cell types can simply be mixed and co-incubated together. Although any method of co-culturing cells known in the art can be used, in a preferred methodology the two cell types are combined and centrifuged to form a pellet. The pellet is then diluted with a culture medium, preferably RPMI or AIM 5, and incubated overnight at about 37° C. in a 5% CO₂ incubator. As with the hybridoma formulation, any ratio of DC to afflicted cell can be used; a ratio of between about 1:100-100:1 is preferred, a ratio of between about 1:10-10:1 is more preferred and the ratio of about 6:1 is most preferred. It will be appreciated by one skilled in the art that the co-culture product can be used in the methods of the present invention without a selection step.

The terms “products of co-culture” or “co-culture product(s)”, as used herein, refer to matter resulting from co-culture of afflicted cells and DCs and can include, for example, tumor cells and dendritic cells which have become fused together, dendritic cells which have internalized or become associated with antigenic tumor components or other components of tumor cells, tumor cells which have internalized or become associated with dendritic cell components relevant to antigen-presentation function, or subcellular components derived from any of the cells described above. It will be understood, therefore, that these terms reflect that during co-culture a stimulator cell complex is produced which contains tumor antigen and molecules necessary for antigen presentation to T-cells. The same applies to use of virally infected cells.

Following preparation of either the fusion product or the co-culture of DCs and afflicted cells, T-cells from the patient should then be added to the dendritic cell/afflicted cell combination. The T-cells, like the DCs and, in a preferred embodiment the afflicted cells, should all derive from the same patient. Thus, the DCs, afflicted cells, and T-cells are preferably all autologous, and should be obtained from the patient who will ultimately be treated with the T-cells generated according to the present methods. The use of autologous cells eliminates the need to identify and characterize the specific antigens unique to various tumor cells and virally infected cells, which can vary from patient to patient and from illness to illness. The T-cells prepared according to the present methods have the capability of targeting the particular antigens generated by each host, since they themselves have been co-cultured with those antigens. In addition, if using allogenic tumor cells, “shared” antigens will be present in the co-culture.

The term “T-cell” as used herein will be understood by those skilled in the art. The term includes, but is not limited to CD8⁺ or CD4⁺ T-cells capable of lysing target cells or providing effector or helper functions, such as cytokine secretion, which can result in the death of target cells or the generation or enhancement of anti-target effector activity. The invention is not intended to be limited to these examples, however, and any T-cells known in the art can be used according to the present invention. Preferably, the T-cells are unstimulated T-cell precursors.

T-cells can be obtained from any suitable source, including, but not limited to spleen tissue, lymph nodes, peripheral blood, tumors, ascitic fluid, dermal biopsies, and CNS fluids. Any method for harvesting T-cells from the host can be used. For example, Ficoll-Paque (commercially available from Pharmacia) centrifuged peripheral blood mononuclear cells (PBMC) can be used. Alternatively, purified CD4⁺ or CD8⁺ T-cells isolated by immunoaffinity procedures, such as through the use of MACs or dyna-beads, can be used. Appropriate methods for obtaining T-cells are taught, for example, in Tüting, et al., J. Immunol. 160: 1139-1147 (1998).

The harvested T-cells should then be added to the media containing the fusion product or co-culture product of the dendritic cells and afflicted cells. A ratio of 10:1 to 100:1 T-cells:dendritic cells is preferred, although any other suitable ratio is also within the scope of the invention.

The mixture of the T-ells, dendritic cells, and either tumor cells or virally infected cells should then be cultured. The cells can be cultured for seven to ten days in culture medium with restimulation at seven to ten-day intervals with identically prepared DC/afflicted cell stimulators. Cultures may be maintained indefinitely using this protocol. Culture medium is preferably RPMI or AIM-V medium supplemented with FCS or HABS (0-10%) and interleukin-2 (5-100 IU/ml). Approximately five to ten days after the last stimulation, T-cells are analyzed for cytotoxicity and cytokine-release assays, or after three to five days from the last stimulation for proliferation-based assays.

Harvesting the T-cells from the co-culture can be accomplished by any means known in the art. For example, harvesting can be simply accomplished by pipet transfer of cultured cells from the flask or plate into a centrifuge tube. After centrifugation, cells are recovered from the pellet and used in the readout assays (i.e., cytotoxicity, cytokine release and proliferation assays). More specifically, CD4⁺ and CD8^{′} T-cells can be selectively recovered using the immunoaffinity procedures discussed above. Typically, after 2-3 rounds of in vitro stimulation only T-cells are in the cultures. The T-cells can be used in any variety of manners, including in methods for treating patients, as probes for specific antigens, and in methods of establishing animal models useful in the study of the immunological arts.

The present invention is therefore also directed to a method of effecting immunotherapy in a host comprising combining dendritic cells from a patient with afflicted cells in vitro; adding unstimulated T-cell precursors from the patient to the dendritic cell/afflicted cell combination; co-culturing the mixture; harvesting the T-cells from the mixture; and administering to a mammalian host an effective amount of harvested T-cells. As used herein, the terms “host”, “mammalian host” and “patient” refer to the organism from which the relevant cells are being extracted and/or who is being treated by the present methods, including but not limited to humans. It will be understood that “immunotherapy” includes the prophylactic treatment of a patient susceptible to tumors, such as a patient in a high risk group for certain types of cancer; “immunotherapy” also includes the therapeutic treatment of a tumor-bearing patient. DCs and tumor cells as described above can be used. It will be further appreciated that the type of tumor cell used will depend on the type of cancer for which the treatment is being administered. For example, if the patient is being treated to provide prophylactic resistance to melanoma, melanoma cells should be used. The patient's own melanoma cells, or allogenic melanoma cells containing shared antigens, should be used. “Immunotherapy” also includes both the prophylactic treatment of a patient prior to a viral infection and the therapeutic treatment of patients having a viral infection. Any DCs and virally infected cells as described above can be used. Again, the type of virally infected cell used will vary depending on the viral infection for which treatment is being provided.

An effective amount of the T-cells generated according to the present methods should be administered to the host being treated. As used herein, the term “effective amount” refers to that amount of T-cells to achieve the desired immunotherapy, such as the amount of T-cells which will bring about the desired level of prophylactic resistance or therapeutic relief to a patient.

As will be appreciated by those skilled in the art, the effective amount will differ from patient to patient depending on such variables as whether the use is prophylactic or therapeutic, the size and/or severity of the tumor or tumors, the type and/or severity of the viral infection, the size and weight of the patient, and the like. Even a minimal dosage of antigen-specific T-cells would provide a benefit to a patient. It is within the skill of one practicing in the art to determine the effective amount for each patient; a typical maximum dosage per treatment would be about 1011 T-cells. Higher or lower doses can be used depending on the patient being treated. The number of treatments will also depend on the patient being treated, the illness being treated, the patient's response to treatments, and the like. Again, it is within the skill of the practitioner to determine the appropriate number of treatments.

The T-cells can be introduced to the host by any means known in the art including but not limited to the use of a pharmaceutical composition prepared with the T-cells. For example, the T-cells can be combined with a suitable pharmaceutical carrier. Any suitable pharmaceutical carrier can be used, as long as compatibility problems do not arise. The preferred carriers are saline, phosphate buffered saline (PBS) and media which contains T-cell growth factors. The T-cell compositions can be administered, for example, intravenously, intramuscularly, intralymphatically, intradermally, subcutaneously and intratumorally.

The T-cells of the present invention can also be utilized to identify tumor or viral antigens. It will be appreciated that when tumor cells are used to stimulate T-cells, the T-cells will be useful in the identification of tumor antigens, and when virally infected cells are used to stimulate T-cells the T-cells will be useful in identifying viral antigens. For example, T-cells may be used as indicator reagents to identify specific tumor peptides or tumor gene products, as taught by Storkus et al., J. Immunol. 151: 3719-3727 (1993) and van der Bruggen et al., Science 254: 1643-1647 (1991). Antigen presenting cells (such as dendritic cells or transformed cell lines) may be loaded with fractionated peptides extracted from tumor cells (Storkus et al., 1993) and then analyzed for reactivity (cytotoxicity, proliferation, cytokine release, etc.) with T-cells prepared according to the current invention. Those peptide pools recognized by T-cells may then be analyzed using mass spectroscopy or Edman degradation sequencing methods to identify the sequence of individual peptides within the pool. Synthetic peptides conforming to these sequences may then be generated and tested for T-cell reactivity, with positively recognized peptides constituting potential vaccine components. Alternatively, tumor-derived DNA or cDNA may be transfected into DC or transformed cell lines (van der Bruggen, 1991) and the resulting transfectants screened for their ability to be recognized by T-cells obtained by the methods of the current invention. Transfected DNA extracted from targets recognized by T-cells may then be sequenced, with the resulting tumor-associated gene/gene product constituting a potential gene vaccine component. In addition, peptides derived from these gene products may also serve as components of peptide-based vaccines and therapies.

T-cells can also be generated according to the present methods for use in preparing animal models. Such models would be useful, for example, in studying the various effects of various types of immunotherapy on the host being treated. For example, T-cells may be induced in vivo in mice as outlined by Celluzzi and Falo, J. Immunol. 3081-3085 (1998), with tumor vaccination and therapy being the outcome. Alternatively, using the current invention, antigen-specific T-cells may be generated ex vivo, with the expanded cells adoptively transferred into tumor bearing mice or patients, or virally-infected patients. Since murine models allow for both protective and therapeutic systems to be evaluated, this approach serves to define surrogate systems in which to validate the potential clinical efficacy of various treatment methodologies.

When using the present T-cells, the immunotherapy of the present invention results in the induction of tumor specific lytic activity in an immunized mammalian host. That is, prophylactic or therapeutic treatment will be specific for the type of tumor used in the DC-tumor cell hybridoma or co-culture. Such immunization protects patients from tumor challenge and/or results in regression of established tumors. Similarly, prophylactic or therapeutic treatment will be specific for the type of virus used, and will protect patients from viral challenge and/or will result in reduction of viral infection. Thus, the present methods of immunotherapy eliminate the need to isolate and characterize individual antigens. The present invention therefore provides a rapid, inexpensive and efficient technology for generating antigen-specific T-cells in vitro. These T-cells can be used as immunotherapy through the adoptive transfer of autologous antigen-specific T-cells in patients suffering from tumors or viral infections.

### EXAMPLES

The following examples are intended to illustrate the invention, and should not be construed as limiting the invention in any way. The mice used in the examples were female C57BL/6 mice, 5-8 weeks old and were obtained from the Jackson Laboratory in Bar Harbor, Me. B16 is a C57BL/6 melanoma (H-₂b) obtained from ATCC, Rockville, Md., and 3LL is a lung carcinoma also available from ATCC. Cell lines were maintained in DME containing 10% FCS and antibiotics. Monoclonal antibodies used to deplete cell subsets were prepared from the hybridomas GK 1.5 (anti-CD4, ATCC TIB 207), 2.43 (anti-CD8, ATCC T1B210), 30-H12 (anti-Thy 1.2, ATCC T1B107), B220 (anti-B cell surface glycoprotein, ATCC T1B 146), and NK1.1, obtained from W. Chambers, University of Pittsburgh School of Medicine.

### Example 1

### Fusion of DCs and Tumor Cells

Dendritic cells were prepared from bone marrow as generally described in Celluzzi et al., J. Exp. Med. 183: 283-287 (1996) using GM-CSF as described in the reference. Briefly, bone marrow cells were depleted of lymphocytes and cultured at 5×10⁵ cells/ml in 10% FCS-containing RPMI 1640, obtained from Irvine Scientific, Santa Ana, Calif., with granulocyte macrophage-colony stimulating factor (GM-CSF), in a concentration of 103 U/ml, obtained from Sigma Chemical Company, St. Louis, Mo. Loosely adherent cells were collected on day 6 for fusion. Between about 50 and 75% of the DCs expressed CD86 (B7.2) and Class II MHC (I-A⁺) antigens, as determined by flow cytometry.

Day 6 DCs were fused with either B16 or 3LL cells at a ratio of 6:1, DC to tumor cells, using polyethylene glycol at 37° C. After washing by centrifugation, fused cells were cultured overnight at 37° C. in RPMI 1640 (10% FCS).

### Example 2

### Preparation of Dendritic Cell and Tumor Cell Co-cultures

Dendritic cells were prepared according to the methods of Example 1. Day 6 dendritic cells were then used to form a co-culture with either B16 cells or 3LL cells. Each DC/tumor cell co-culture was prepared by placing DCs into test tubes with the respective tumor cells. A pellet of cells was formed by centrifugation. The pellet was then diluted with RPMI (10% FCS) and incubated overnight at about 37° C. in a 5% CO₂ incubator. The ratio of DCs:tumor cells was about 6:1. The products of the co-culture were prepared to further evaluate whether tumor antigens were present in close association with DCs and to evaluate if soluble factors released from the tumors were present on the DCs.

### Example 3

### Efficiency of Fusion and Co-Culture

To determine the efficiency of fusion and co-culture, each of the cell types—DCs, B16 and 3LL—was stained with a different lipophilic fluorochrome before fusion and analyzed using flow cytometry. The tumor cells were stained with DiO while the DCs were stained with Dil, both of which were obtained from Molecular Probes, Inc., Eugene, Oreg. After extensive washing, cells were fused or co-cultured and allowed to incubate overnight at 37° C. Harvested cells were then fixed in 2% paraformaldehyde and the forward and side scatter patterns measured on a Becton Dickinson Facstar Plus with Argon/HeNe duel laser, available from Becton Dickinson Immunocytometry Systems, San Jose, Calif.

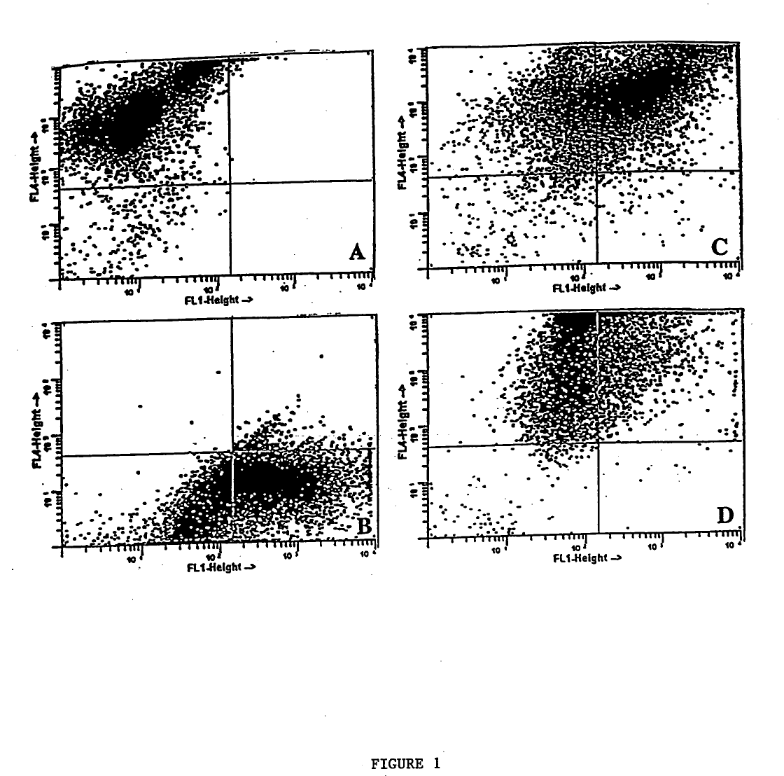

The scatter pattern of each cell type is depicted in FIG. 1. Individual cell staining shows two distinct patterns of DCs (DiI) which shift up (upper left quadrant of FIG. 1A) or B16 tumor cells (DiO) which shift right (lower right quadrant of FIG. 1B) as compared to unstained controls (not shown). When cells were co-cultured (FIG. 1C) or fused (FIG. 1D), the patterns shifted both to the right and up, indicating that those cells were doubly stained. This upper right quadrant was used as the indicator of the “efficiency” of the association of tumor antigens with dendritic cells. Cells found in the upper left or lower right quadrants were regarded as singly stained cells and were not included in the measure. By these standards, the association efficiency was quite high, ranging from about 70% in the co-cultured group to about 53% in the fusion group of the total gated cells.

### Example 4

### Preparation of Antigen-Specific T-Cells

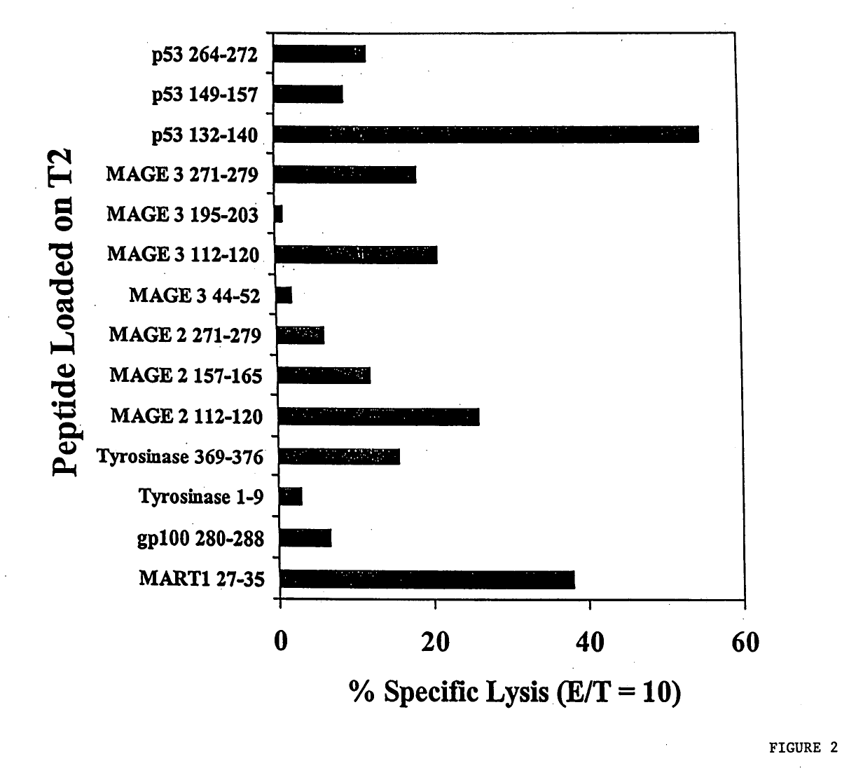

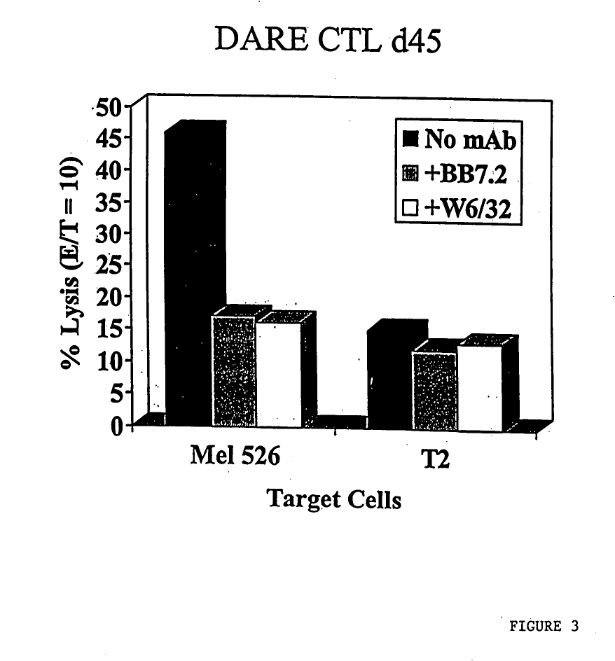

Melanoma was resected from a human HLA-A2+ patient, digested to produce a single cell suspension, and cryopreserved. Dendritic cells were derived from peripheral blood by culture in AIM V medium containing rhIL4 and rhGmCSF, following the procedure generally set forth by Tüting, et al., J. Immunol., 160: 1139-1147 (1998). At day 7, tumor cells were thawed, irradiated (15,000 rad), and added to the seven day DC culture. The tumor cells and DCs were allowed to “co-incubate” for 24 hrs at 37° C. The DC:tumor cell ratio was approximately 10:1. Co-cultured cells were harvested by pipette and irradiated at 3,000 rads. Harvested cells were added to T-cell cultures as stimulators on a weekly basis at a ratio between about 10:1 and 100:1 (T-cells:DCs). After 45 days T-cells were assessed for their ability to lyse tumor peptide pulsed T2 cells (FIG. 2) or tumor cell line targets (FIG. 3). Lysis in the cytotoxic assay was performed using standard procedures. Targets were HLA-A2 matched allogeneic melanoma targets (i.e., Me1526). FIG. 2 demonstrates that T-cell lines not normally recognized can become recognized when loaded with peptides that derive from proteins expressed by melanoma cells (such as MART-1, gp100, tyrosinase, etc.). The results illustrated in FIG. 2 demonstrate that the DC-tumor stimulators of the present invention can be used to drive the expansion of T-cells that recognize a wide array of tumor-associated antigens. The same concept would apply with DC-virally infected cell stimulators against viral associated antigens. Thus, the T-cells of the present invention have a broad range of reactivity; that is particularly applicable in a clinical setting, since tumor cells or virally infected cells may attempt to modulate their expression of individual antigens based on immune selective pressure in vivo. FIG. 3 demonstrates the results obtained when the T-cells of the present invention were evaluated for their ability to lyse an HLA-A2 matched melanoma target (Me1526). The T-cells killed the target effectively; this killing could be blocked by antibodies that bind to the HLA-2 molecule (BB7.2 and W6/32), demonstrating the HLA-A2 class 1 restriction of T-cells in these expanded T-cell cultures. The T-2 cell line is an HLA-A2 matched target. The results demonstrated in the figures therefore support HLA-A2 restricted CTL activity directed against both intact melanoma and defined peptide epitopes presented by HLA-A2.

Whereas particular embodiments of this invention have been described above for purposes of illustration, it will be evident to those skilled in the art that numerous variations of the details of the present invention may be made without departing from the invention as defined in the appended claims.

1. A method for generating antigen-specific T-cells, comprising:
a) combining at least one first cell with at least one second cell in vitro, wherein said first cell is an autologous dendritic cell and said second cell is selected from the group comprising a tumor cell and a virally infected cell;
b) adding autologous T-cells to the combination of step a);
c) culturing the mixture of step b); and
d) harvesting the T-cells from the mixture of step c).

2. The method of claim 1, wherein said dendritic cells are selected from the group comprising cutaneous epidermal Langerhans cells, dermal dendritic cells, lymph node dendritic cells, spleen dendritic cells, dendritic cells derived through in vitro culture of precursors and blood-derived dendritic cells.

3. The method of claim 1, wherein said second cell is selected from the group comprising autologous cells and allogenic cells.

4. The method of claim 1, wherein said tumor cells are selected from the group comprising melanoma cancer cells, lung cancer cells, prostate cancer cells, breast cancer cells, colon cancer cells and cervical cancer cells.

5. The method of claim 1, wherein said virally infected cells are selected from the group comprising cells infected with influenza virus, human immunodeficiency virus, cytomegalovirus, human papilloma virus and herpes simplex virus.

6. The method of claim 1, wherein said first cell and second cell are fused to create a hybridoma.

7. The method of claim 6, wherein said hybridoma contains a ration of first cells to second cells between about 1:100 and 100:1.

8. The method of claim 6, wherein said hybridoma contains a ratio of first cells to second cells of about 6:1.

9. The method of claim 1, wherein said first cell and second cell are co-cultured.

10. The method of claim 9, wherein said co-culture contains a ration of first cells to second cells between about 1:100 and 100:1.

11. The method of claim 9, wherein said co-culture contains a ratio of first cells to second cells of about 6:1.

12. The method of claim 1, wherein said T-cells are added in a ration of between about 10:1 and 100:1 T-cells to dendritic cells.

13. The method of claim 1, wherein said T-cells are unstimulated T-cell precursors.

14-36. (canceled)
